(19) United States
(12) Patent Application Publication
(10) Pub. No.: US 2021/0259894 A1
(43) Pub. Date: Aug. 26, 2021

(54) Surgical Gauze

(71) Applicant: City of Hope, Duarte, CA (US)

(72) Inventor: Gagandeep Singh, Duarte, CA (US)

(21) Appl. No.: 17/184,987

(22) Filed: Feb. 25, 2021

Related U.S. Application Data
(60) Provisional application No. 63/080,155, filed on Sep. 18, 2020.
(60) Provisional application No. 62/981,392, filed on Feb. 25, 2020.

(51) Int. Cl. A61F 13/44

(52) U.S. Cl. CPC A61F 13/44; A61B 2090/3954

(57) ABSTRACT

A surgical material, such as gauze, includes a feature that increases the magnetic permeability of the surgical material. The surgical material incorporates one or more elements with high magnitude of magnetic permeability within the surgical material. In an embodiment, the one or more elements are coupled to a strip of material such as in the form of beads.

### CROSS REFERENCE TO RELATED APPLICATIONS

This application claims priority to U.S. Provisional Patent Application No. 63/080,155 filed Sep. 18, 2020, entitled “Surgical Gauze”, and U.S. Provisional Patent Application No. 62/981,392 filed Feb. 25, 2020, entitled “Surgical Gauze”, the contents of which are hereby incorporated by reference in their entirety and for all purposes.

### BACKGROUND

It is uncommon for a surgeon to accidentally leave surgical materials in a patient's body after a surgical operation such that the surgical material is retained in the body after surgery. This often occurs despite precautionary procedures, such as the fact that the manual counts of surgical materials done in the operating room are reported to be correct. One of the most common material left in the body is surgical gauze. A retained gauze or lap pad is referred to as gossypiboma. The negative results for both the patient and the surgeon can be grave.

In order to reduce the likelihood of incidences of gossypiboma, many types of surgical gauze are implanted with or otherwise contain materials that can be detected by x-rays or CT scans. Such types of gauzes have increased likelihood of being detected via x-rays or CT scans during a surgical procedure to reduce the risk of gossypiboma. Unfortunately, there are currently no gauzes or other similar devices (such as surgical lap pads) that are configured to be detected by magnetic resonance imaging (MRI). Thus, a lost gauze pad or item such as a lap pad cannot be detected if a patient is only getting an MRI for surveillance and detection.

In view of the foregoing, there is a need for improved devices, systems and methods for preventing gossypiboma via Mill.

### SUMMARY

Disclosed is a surgical material, such as a gauze material and/or a lap pad (in non-limiting examples), that is configured to be detected or otherwise sensed by any of a variety of imaging modalities including at least MRI. In optional embodiments, the surgical material can also be detected by other imaging modalities such as ultrasound, x-ray and computer tomography (CT) scans.

In an example embodiment, the surgical material, such as gauze, includes a feature that increases the magnetic permeability of the gauze entirely or partially by entirely or partially incorporating one or more elements with high magnitude of magnetic permeability within the surgical material. The one or more elements with high magnitude of magnetic permeability can be coupled to the surgical material in a variety of manners. Such elements can be diamagnetic or paramagnetic with a magnetic permeability magnitude higher than a predetermined value, such as a magnetic permeability magnitude higher than 0.05 or higher than 0.08 (in nonlimiting examples.)

As described in more detail below, the gauze can incorporate a material such as water in the form of beads. In another embodiment, the gauze can incorporate beads that contain mineral oil, gadolinium-based contrast agents, and/or other chemicals. In addition, or as an alternative, the gauze can include one or more barium strips of water or oil embedded into the strips such as in a bead form. The incorporation can vary. For example, the material can be embedded within the gauze or can be positioned at least partially external or internal to the gauze.

In one aspect, there is disclosed a surgical material for use in surgery, comprising: a planar material configured to be used in a surgical procedure; and a detectable element coupled to the planar material, wherein the detectable element is detectable via magnetic resonance imaging (MRI).

The details of one or more variations of the subject matter described herein are set forth in the accompanying drawings and the description below. Other features and advantages of the subject matter described herein will be apparent from the description and drawings.

### BRIEF DESCRIPTION OF THE DRAWINGS

FIG. 1 shows a schematic representation of a surgical material that includes an element that is detectable by an MRI.

FIG. 2 shows a schematic representation of a gauze material having a strip of MRI detectable material.

FIG. 3 shows an example configuration of such a strip containing a series of beads.

### DETAILED DESCRIPTION

Disclosed are systems and methods comprising a surgical material, such as a gauze material and/or a lap pad, that is configured to be detected or otherwise sensed by any of a variety of imaging modalities including at least MRI. In optional embodiments, the surgical material can also be detected by other imaging moralities such as x-ray and CT scans. The surgical material is sometimes described herein in the context of being gauze although it should be appreciated that the surgical material can vary. In another example embodiment, the surgical material is a lap pad, which can be made of various materials including cloth.

Gauze is a loose, openly woven surgical dressing made from a material such bleached cotton that is used in a variety of wound care applications. Often used for wound dressings, bandages and absorbent sponges, gauze is available in a variety of forms, such as bulky gauze pads, rolls and sponges. Gauze often comes in a planar format such as in the form of a pad. The gauze is described herein in the context of being a pad or a planar element although it should be appreciated that the format of the gauze, including the shape, density, structure, and material can vary.

As mentioned, conventional surgical gauze is not detectable by MR, which provides a main magnetic field via a large electric current flowing through wires that are formed into a loop of a magnet of the imaging system. A typical clinical MRI system includes a magnetic field strength of about 1.5 to 3.0 T (Tesla). The magnetic field polarizes the magnetic dipoles of the atoms that are being detected either along a direction of the magnetic field or against the direction of the magnetic field. This can vary depending upon whether the atom is para or ferro magnetic or diamagnetic. When a radiofrequency signal is superimposed on the main magnetic field it causes a change in the direction of the magnetic field. As the atoms respond to such a change in direction of the magnetic field, the sensor detects the changing magnetic field. Relaxation times are different depending upon the magnetic properties of the objects. Therefore, by specially mapping the relaxation times, objects with different magnetic field properties can be imaged.

An ability of the MRI to detect objects at least partially depends on the magnetic properties of the atoms being detected. A material magnetic permeability very close to zero, for example, does not respond to the changing magnetic field and is thus not visible under MRI. It is been observed that water has a magnetic permeability of 0.09, which provides sufficient diamagnetism to be detected by MRI. This is because water is diamagnetic and aligns itself against the magnetic field. Water therefore responds to the changing magnetic field depending upon a water content of tissue being detected. Different tissues have different relaxation times and therefore can be imaged using an MRI.

A conventional surgical gauze is generally formed of cotton. A material such as cotton typically has a magnetic permeability that is lower magnitude than that of water. As a result, an MRI machine cannot detect the presence of gauze such as the presence of gauze in the body. This has implications in monitoring any complications from retained gauze or lap pads within the patient after surgery.

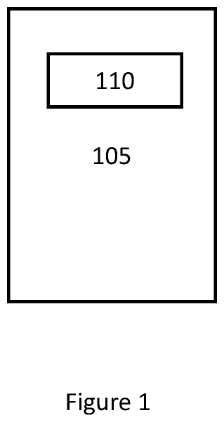

FIG. 1 shows a schematic representation of a surgical material 105 such as gauze. The surgical material 105 includes, is embedded with, contains, is attached to, or is otherwise coupled to an element 110 that is configured to be detected by MRI. The surgical material 105 is represented as a rectangular shape although it should be appreciated that the size, shape, and structure of the surgical material can vary. The size, shape, and structure of the element 110 can also vary. In an embodiment, the surgical material is at least partially woven and/or is a planar structure.

The element 110 is configured to alter or increase the magnetic permeability of the surgical material 105 entirely or at least partially such as by incorporating or at least partially including a material with a high magnitude of magnetic permeability. As mentioned, in an example embodiment, the element 110 is diamagnetic or paramagnetic with a magnetic permeability magnitude higher than 0.05 or higher than 0.08 in nonlimiting examples. In a nonlimiting example, the element 110 at least partially includes water.

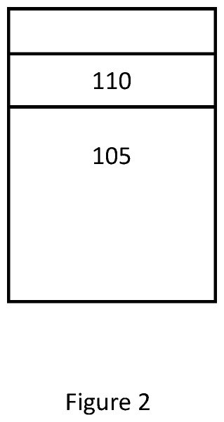

FIG. 2 shows a piece of gauze 205 having a strip 110 of material, where the strip 110 is at least partially made of a material that is configured to be detected by MRI. The strip 110 can comprise a thread or collection of threads in an example embodiment. The strip 110 can be entirely made of an MRI detectable material or it can be partially made of an MRI detectable material. The strip 110 is an elongated body, which can extend entirely across the gauze from one end to another. Or the strip 110 can extend partially along the gauze such that the strip does not entirely extend from one end to another end of the gauze.

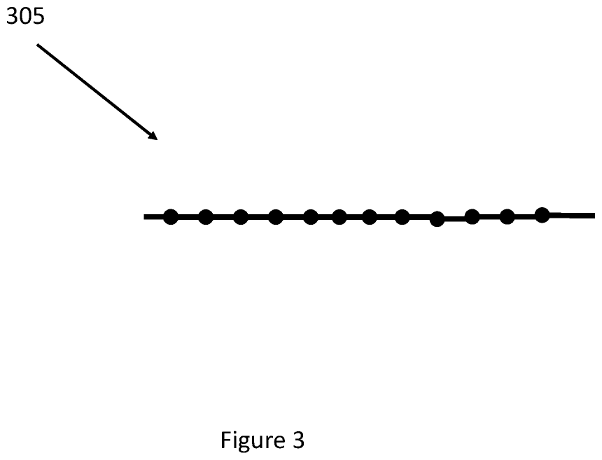

In a non-limiting example, as shown in FIG. 3, the strip 110 can be formed by a series of interconnected beads 305. Each of the beads 305 contains or is at least partially formed of a material configured to be detected by MRI. For example, the beads 305 can be filled with water. In an embodiment, the strip 110 is a tubular structure that is a radiopaque tubing wherein the strip at least partially formed of or includes water. In a non-limiting embodiment, the strip/thread 110 is at least partially formed of a polymer or a chemical compound that combines a metal or chemical that renders it detectable by MRI, X-Ray and CT scan. This encompasses molecules as well.

The beads can vary in configuration. For example, each bead can a different size or shape relative to at least one other bead. The beads 305 in an embodiment are all the same size or shape. The beads can extend along the entire length of the strip or along a portion of the strip. The spacing between the beads can also vary along the strip. In an embodiment, the strip 110 does not have beads.

The material of the strip 110 is compatible with in vitro human environment and has sufficient elastic modulus and thermal stability to withstand any expansion of water during high-temperature sterilization. The strip 110 can be inserted inside the gauze or can be positioned on the surface of the gauze so that the beads and the tubing (if present) will appear in an MRI scan and show the position of the gauze under MRI. In a nonlimiting example, each of the beads is approximately the size of a grain of rice. In another embodiment, each of the beads contains mineral oil or gadolinium-based contrast agent.

In an example embodiment, the gauze 205 includes a wireless identifier, such as a Radio-frequency identification (RFID) or identifier that operates pursuant to another wireless paradigm. The RFID can be positioned on, within, or otherwise coupled to the gauze 205 for providing a wireless identifier and/or locator for the gauze.

In an exemplary method, the gauze 205 is used in a surgical procedure. Pursuant to the procedure, a user is provided with the gauze. The use employs the gauze during the procedure, such as by inserting the gauze on or into the patient prior to, during, or after the procedure. The gauze 205 can be used in any of a wide variety of well-known manners. Pursuant to the surgical procedure, at least one imaging modality is used. In an example, the imaging modality comprises an MRI wherein the imaging modality is used prior to, during, or after the surgical procedure. As mentioned, the gauze 205 is configured to be detected by MRI. In another nonlimiting example, the gauze is further configured to be detected by x-ray or CT scan. Pursuant to the method, the imaging modality detects the presence of the gauze 205 during at least a portion of the surgical process, prior to the procedure, or after the procedure. In an embodiment, a system or kit includes a device configured to perform an imaging process, such as an x-ray device, a CT device, an MRI device or any imaging device, and further includes a surgical gauze as described herein.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A surgical material for use in surgery, comprising:
a planar material configured to be used in a surgical procedure;
a detectable element coupled to the planar material, wherein the detectable element is detectable via magnetic resonance imaging (MRI).

2. A surgical material as in claim 1, wherein the planar material is gauze.

3. A surgical material as a claim 1, wherein the detectable element is an elongated strip of material.

4. A surgical material as a claim 3, wherein the strip of material includes a series of beads.

5. A surgical material as a claim 4, wherein each of the beads includes a material that is detectable by Mill.

6. A surgical material as a claim 5, wherein the material that is detectable by MRI is a liquid.

7. A surgical material as a claim 5, wherein the material that is detectable by MRI is water.

8. A surgical material as a claim 4, wherein at least one of the beads comprises a magnetic permeability of higher than 0.05.

9. A surgical material as a claim 1, wherein the detectable element is further detectable by an imaging modality other than Mill.

10. A surgical material as a claim 9, wherein the detectable element is further detectable by at least one of x-ray and CT scan.

11. A method of using a surgical material, comprising:
coupling a surgical material to a patient, the surgical material comprising:
a planar material configured to be used in a surgical procedure;
a detectable element coupled to the planar material, wherein the detectable element is detectable via magnetic resonance imaging (MRI) using an imaging system to detect a presence of the surgical material.

12. The method of claim 11, wherein coupling the surgical material to the patient comprises inserting the surgical material into the patient.

13. The method of claim 11, wherein the planar material is gauze.

14. The method of claim 11, wherein the detectable element is an elongated strip of material.

15. The method of claim 14, wherein the strip of material includes a series of beads.

16. The method of claim 15, wherein each of the beads includes a material that is detectable by MRI.

17. The method of claim 16, wherein the material that is detectable by MRI is a liquid.

18. The method of claim 15, wherein the material that is detectable by MRI is water.

19. The method of claim 15, wherein at least one of the beads comprises a magnetic permeability of higher than 0.05.

20. The method of claim 11, wherein the detectable element is further detectable by an imaging modality other than MRI.
